(19) United States
(12) Patent Application Publication
(10) Pub. No.: US 2009/0054916 A1
(43) Pub. Date: Feb. 26, 2009

(54) CLIP-BASED METHOD FOR TREATMENT OF UTERINE FIBROIDS BY OBSTRUCTION OF THE UTERINE ARTERIES

(76) Inventors: Peter Meier, Hamburg (DE); Burkharo Peters, Wattenbeck (DE); Axel Hentrich, Hamburg (DE)

Correspondence Address: GREENBERG TRAURIG, LLP, 200 PARK AVE., P.O. BOX 677, FLORHAM PARK, NJ, 07932, US

(21) Appl. No.: 11/843,944

(22) Filed: Aug. 23, 2007

(51) Int. Cl. A61B 17/08

(52) U.S. Cl. 606/158

(57) ABSTRACT

A device for degenerating a fibroid comprises a pincher mechanism capable of pinching a uterine artery with a force sufficient to substantially obstruct blood flow therethrough. The pincher mechanism is sized and shaped so as to access the uterine artery through a wall of a patients vaginal vault. In a method for degenerating a uterine fibroid, the aforesaid pincher mechanism is, closed about a uterine artery so as to block the flow of blood therethrough. The pincher mechanism is maintained in the closed position for a period of time-necessary to degrade or kill the fibroid. After such time has, passed, the pincher mechanism is opened, removed from around the artery such that normal blood flow resumes.

### FIELD OF THE INVENTION

This invention relates, generally, to the treatment of uterine fibroids by obstruction of the uterine arteries. More specifically, it relates to the use of mechanical instruments to block the flow of blood through the arteries.

### BACKGROUND OF THE INVENTION

Uterine leiomyomas (i.e., fibroids) are extremely common benign tumors, which are located primarily within the uterine muscle (i.e., intramural fibroids), the uterine cavity (i.e., submucosal fibroids) or on the serosal surface of the uterus. Such fibroids occur in approximately 20% to 30% of women older than 30 years of age. Medical treatment is usually sought when the fibroids are associated with menorrhagia, pelvic pain or urinary symptoms, or when they are suspected to be the cause of infertility. Treatment options include medical therapy and various types of surgical intervention.

Hysterectomy: is considered to be the definitive surgical treatment for those women who no longer wish to maintain their fertility. Though effective, this method has a number of undesirable characteristics. First is the mortality rate for this procedure, which is approximately 30 times as great as the mortality rate for women who have not had hysterectomies. Further adverse effects of hysterectomies include damage to adjacent organs, including removal, of the ovaries, lengthy hospital stays and periods of recovery, and an increased likelihood of cardiac arrest, decreased sexual pleasure, and increases in depression or anxiety. Surgical removal of fibroids without hysterectomy, by any surgical method, presents a risk of recurrence of fibroids or, more often, failure to observe existing fibroids or misidentification of the fibroids that are causing adverse symptoms.

It has been established that fibroids can be treated by non-surgical therapies involving the temporary obstruction of the blood flow within the arteries transporting blood into the uterus. One example of such a treatment is uterine artery embolization (UAE). UAE involves the injection of tiny particles of polyvinyl alcohol (PVA) through blood vessels to block the arteries supplying blood to the fibroids. This blockage of the blood supply causes degeneration of the fibroids leading to their death. However, UAE is performed by radiologists who, typically, are unfamiliar with, practices of gynecological care. As of now, UAE's are performed in radiology suites, which have high installation and operational costs and which, therefore, are generally restricted to major medical centers. Also, however UAE is practiced, the movement of the PVA particles is flow-directed and their distribution is not limited to the arteries that supply the fibroids, but may affect blood flow to other areas of the uterine tissue or to the ovaries.

There exists a need for devices and methods that can be used to temporarily obstruct the flow of blood to fibroids. The devices should be relatively inexpensive and simple to apply, and should allow the physician to control the degree by which blood flow is reduced. Various devices and methods for obstructing the uterine arteries have been disclosed in the prior art.

U.S. Pat. No. 6,254,601 discloses methods for penetrating the wall of the vaginal vault near the uterine artery with devices that sense the locations of the anatomical structures and occlude the uterine artery. A number of methods and devices are disclosed. These disclosures are also presented in U.S. Pat. Nos. 6,602,251 and 6,764,488.

U.S. Pat. No. 6,550,482 discloses a clamp for temporarily obstructing the uterine artery. The clamp stretches the wall of the vaginal vault around the artery and applies pressure to stop blood flow.

U.S. Patent Publication No. 2002/0165579 discloses a compression device for distending the wall of the vaginal vault and thus, compressing the uterine artery. Doppler ultrasound techniques are used to locate the uterine artery and sense when blood flow has stopped.

U.S. Patent Publication No. 2002/0183771 discloses a compression device that clamps around the uterine artery and, the vaginal wall to stop blood flow.

U.S. Patent Publication No. 2002/0188306 discloses a forceps-type clamp that is inserted into the vagina and clamps around the uterine artery and; the vaginal wall. Ultrasound sensors are placed on the ends of the clamp to allow location of the uterine artery and sense blood flow. Similar forceps-type clamps are described in a number of other references.

U.S. Patent Publication No. 2002/0124853 is directed to a method of temporarily obstructing blood flow through the uterine artery for a set period of time, then re-establishing blood flow through the artery. A forceps-type clamp is used to compress the artery from both sides.

U.S. Patent Publication No. 2004/0092979 discloses a device with paddles that are used to distend the wall of the vaginal vault around the uterus, thus compressing both uterine arteries at the same time.

U.S. Patent Publication No. 2003/0120286 discloses a clip for encircling and compressing a body lumen, of which a uterine artery is one example.

U.S. Patent Publication No. 2004/0097962 discloses constriction devices that can be deployed to distend the vaginal wall around the uterus and thus obstruct the uterine arteries.

### SUMMARY OF THE INVENTION

The invention provides a device for degenerating a fibroid by using clip-like devices to obstruct the flow of blood through a uterine artery. The invention further provides a method for degenerating a fibroid using a pincher mechanism having two opposable pincher members. The pincher members are placed on opposite sides of a; uterine artery and moved toward each other so as to pinch the uterine artery with sufficient force to substantially obstruct the flow of blood through the artery for a long enough time to degrade the fibroid.

It should be understood that the embodiments described above are merely exemplary and that additional embodiments may be realized that are within the scope of the invention. The invention is further described in the Detailed Description of the Invention presented below.

### BRIEF DESCRIPTION OF THE DRAWINGS

For a more complete understanding of the present invention, reference is made to the following detailed description of the present invention considered in conjunction with the accompanying drawings, in which:

FIG. 1 is a front perspective illustration of an arterial clip according to an embodiment of the present invention.

FIG. 2A is affront view of a component of the arterial clip of FIG. 1.

FIG. 2B is a side view of the component of FIG. 2A.

FIG. 2C is a front view of the component of FIG. 2A in stalled in the arterial clip of FIG. 1.

FIG. 3A is an illustration of the arterial clip of FIG. 1 in its first closed position.

FIG. 3B is an illustration of the arterial clip of FIG. 1 in its first open position.

FIG. 3C is an illustration of the arterial clip of FIG. 1 in its second closed position.

FIG. 3D is an illustration of the arterial clip of FIG. 1 in its second open position.

FIG. 4 is an illustration of the arterial clip, of FIG. 1 in its first open position straddling a uterine artery.

FIG. 5 is an illustration of the arterial clip of FIG. 1 in its second closed position obstructing blood flow through the uterine artery.

FIG. 6 is an, illustration of the arterial clip of FIG. 1 in its second open position once again straddling the uterine artery.

FIG. 7 is an exploded perspective view of an arterial clip according to another embodiment of the present invention.

FIG. 8 is an illustration of the arterial clip of FIG. 7 in its open position.

FIG. 9 is an illustration of the arterial clip of FIG. 7 in its closed position.

FIG. 10 is an illustration of the arterial clip of FIG. 7 being handled with forceps.

FIG. 11 is an illustration of the arterial clip and forceps of FIG. 10, the arterial clip being shown in its open position straddling a uterine artery.

FIG. 12 is an illustration of the arterial clip of FIG. 11 in its closed position obstructing the uterine artery after the removal of the forceps.

FIG. 13 is an illustration of the arterial clip of FIG. 12 being disassembled so as to release the uterine artery.

### DETAILED DESCRIPTION OF THE INVENTION

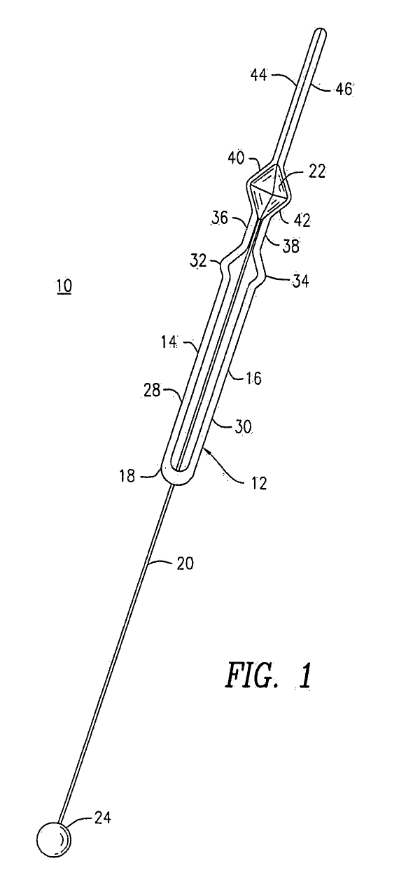

FIG. 1 shows an exemplary arterial clip 10 comprising a generally U-shaped body 12 having a pair of pincher members (“branches”) 14, 16 joined by a U-bend 18. The branches 14, 16 are symmetrical to each other and substantially parallel to, a longitudinal axis through the midpoint of the U-bend 18. The arterial clip 10 also comprises a rod 20 having a solid body 22 having a regular shape (hereinafter referred to as an “expander”) attached eat one end thereof, and a grippable handle 24 attached at the opposite end thereof. The rod 20 extends through the U-bend 18 of the U-shaped body 12 along the aforesaid longitudinal axis, in such a way that the expander 22 is positioned between the branches 14, 16.

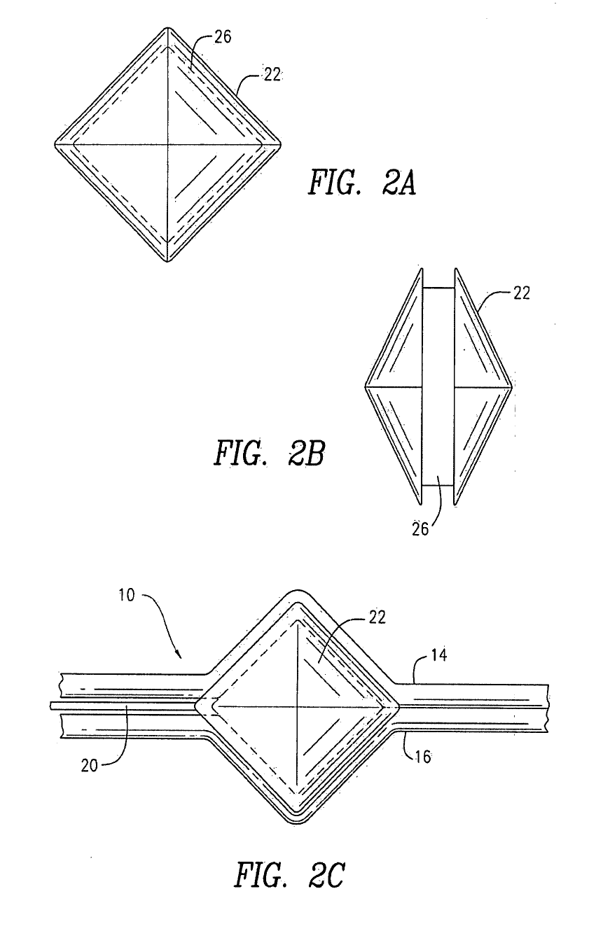

Referring to FIGS. 2A and 2B, it can be seen that the expander 22 is provided with an indentation 26 along its perimeter. The indentation 26 is shaped to accept the branches 14, 16, as can be seen in FIG. 2C. Alternatively, the expander 22 may have a raised edge along its perimeter and each of the branches 14, 16 may have a slot along its length to receive the raised edge.

Returning to FIG. 1, each of the branches 14, 16 has the following sections, in succession away from the U-bend 18: a first straight section 28, 30 that is substantially parallel to the longitudinal axis; a first curved section 32, 34 that is bent away from the longitudinal axis, then back toward it so as to form a curve having a concave side facing the longitudinal axis; a second straight section 36, 38 that is substantially parallel to the longitudinal axis and closer to the longitudinal axis than the first straight section 28, 30 is; a second curved section 40, 42 that is bent away from the longitudinal axis, then back toward it so as to form a curve having a concave side facing the longitudinal axis; and a third straight section 44, 46 (hereinafter referred to as a “tine”) that is closer to the longitudinal axis than the second straight section 36, 38 is. Each of the curved sections 32, 34, 40, 42 is shaped so as to snugly accept the expander 22 between the curved section 32 or 40 on the branch 14 and the corresponding curved section 34 or 42 on the branch 16.

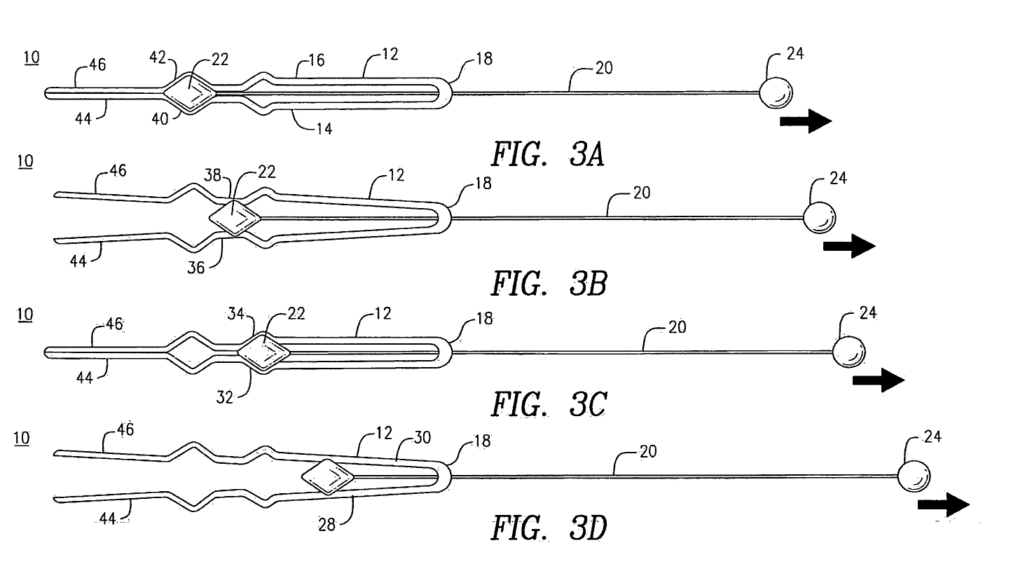

The branches 14, 16 are biased toward each other so that the tines, 44, 46 close against each other when the expander 22 is held between the pair of second curved sections 40, 42, as is shown in FIG. 3A. This position is referred to hereinafter as “the first position” of the expander 22. In FIG. 3B, the expander 22 has been moved toward the U-bend 18 such that it is at a position between the second straight sections 36, 38 (hereinafter “the second position”), causing the tines 44, 46 to separate thereby opening the clip 10. The expander 22 is moved by bracing the body 12 of the arterial clip (e.g., using a tube or forceps, neither of which is shown) and pulling on the rod 20 from the handle 24 end. In FIG. 3C, the expander 22 has been moved to a “third position”, where it is held snugly between the pair of first curved sections 32, 34, allowing the tines 44, 46 to close against each other. Finally, in FIG. 3D, the expander 22 has been moved to a “fourth position”, which is a position between the first straight sections 28, 30, thus separating the tines, 44, 46 of the clip 10.

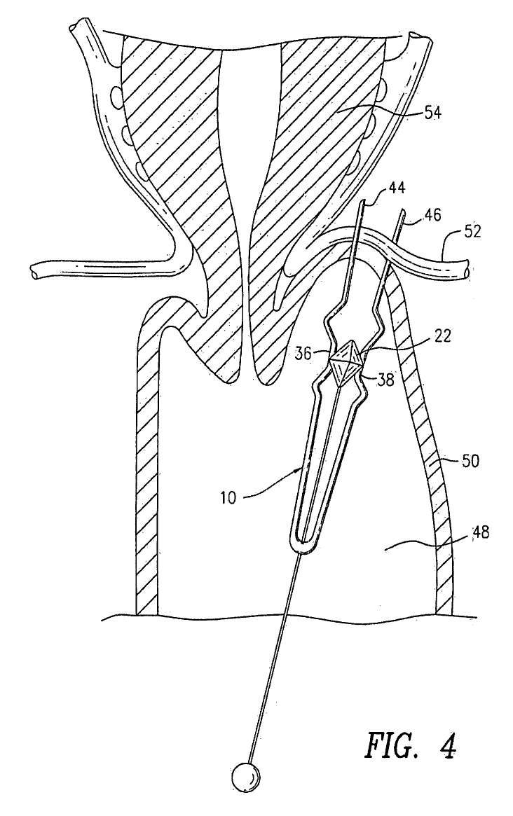

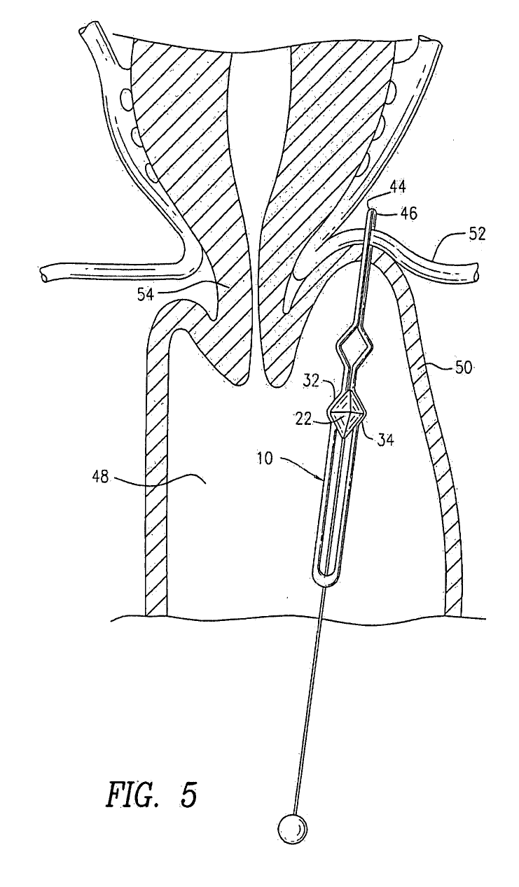

FIGS. 4-6 show steps in the operation of the arterial clip 10. For the purpose of the following disclosure, and for any further disclosures made hereinafter, the relevant features of the female anatomy are the vaginal vault 48, the vaginal wall 50, the uterine artery 52 and the uterus 54.

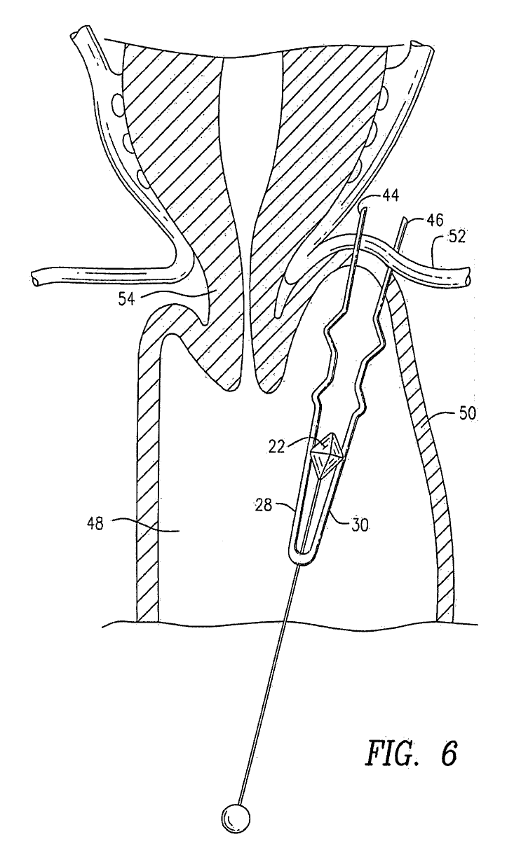

First, an incision (not shown) is made in the vaginal wall 50 and the uterine artery 52 is dissected. As shown in FIG. 4, the expander 22, has been moved to its second position, opening the arterial clip 10. The opened arterial clip 10 is inserted through the incision such that the tines 44, 46 are positioned on either-side of the uterine artery 52. The expander 22 is then moved to its third position, allowing the tines 44, 46 to close, as shown in FIG. 5, thus squeezing the uterine artery 52 shut and obstructing blood flow for a period of time sufficient to degenerate or kill the fibroid without killing the adjacent tissue. Preferably, blood flow through the artery is blocked for 6 to 8 hours, after which the expander 22 is moved to its fourth position, opening the arterial clip 10, as shown in FIG. 6 and allowing normal blood flow to resume. The arterial clip 10 is then pulled back through the incision, using forceps (not shown) or some other gripping device, and the incision is closed.

During the foregoing procedure, it is important that the position of arterial clip 10 be known relative to the uterine artery 52, so that the clip 10 is not mistakenly applied to another blood vessel or to the ureter (not shown). The position of the arterial clip 10 relative to the uterine artery 52 may be determined by any of a number of imaging techniques and/or techniques for monitoring the flow of blood through blood vessels.

Appropriate sensors for imaging and/or blood flow monitoring include blood flow sensors, sound sensors, pressure sensors, or electromagnetic radiation sensors (e.g., X-ray detectors). Sensors may be mounted on the arterial clip 10, on the forceps or other tool used to place or remove the arterial clip 10, or on implements temporarily attached to the arterial clip 10 during insertion. Since any sensor that is used will have associated wiring it is preferable to use an implement that can be removed after the arterial clip 10 is applied to the uterine artery 52. In the absence of such an implement, the patient may move about with the arterial clip 10 in place.

Techniques that may be used include direct visual examination, abdominal ultrasound, Doppler ultrasound, X-ray detection, sound detection, and angiography. Direct visual examination is the preferred method of application. Ultrasound techniques are also beneficial, because they are reliable, real-time techniques for imaging the position of the arterial clip 10 in relation to the uterine artery 52 while the procedure is underway. Doppler ultrasound techniques are especially useful, because they can also be used to determine when blood flow ceases or is restored. Optical fibers may also be used to illuminate the organs, and transmit images to an optical viewer. Adaptations of suitable techniques for use with the arterial clip 10, or other devices that may, be discussed herein, will be apparent to a person skilled in the application of such techniques to surgical procedures.

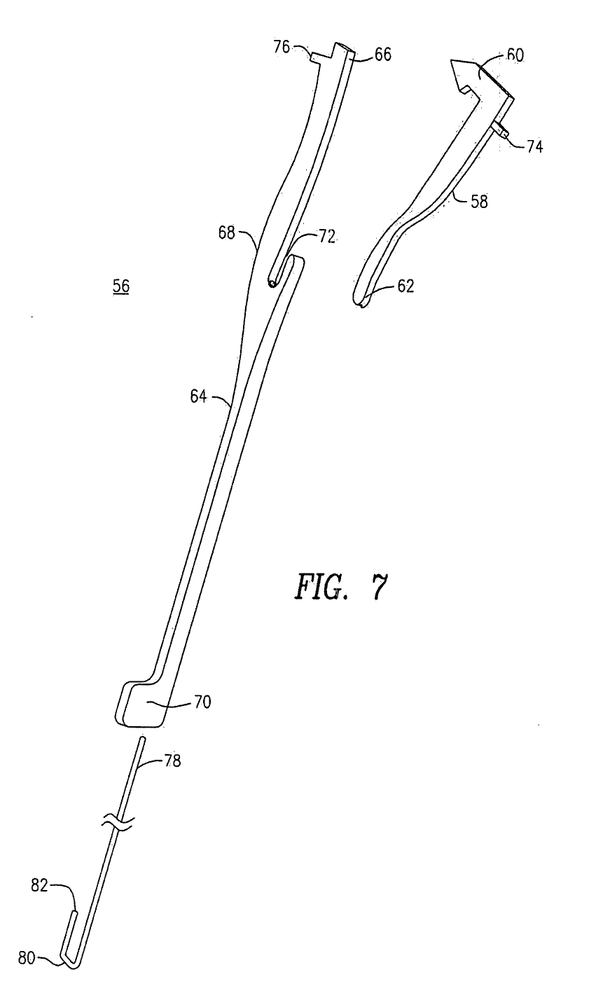

FIG. 7 illustrates an, example of a second arterial clip, 56, in exploded view. The arterial clip 56 comprises three parts. The first part is a short pincher member 58 having a hook 60 at one end and a thin lip 62 at the other end. The short pincher member 58 is made of a bioabsorbable polymer. The bioabsorbable polymers that can be used to make devices according to the present invention include conventional biocompatible, bioabsorbable polymers including polymers selected from the group consisting of aliphatic polyesters, poly(amino acids), copoly(ether-esters), polyalkylene oxalates, polyalkylene diglycolates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarates), absorbable poly(ester urethanes), biomolecules (i.e., biopolymers such as collagen, elastin, bioabsorbable starches, etc.) and combinations and blends thereof. The polyoxaesters include the polymers based on 3,6-dioxaoctanedioic acid, 3,6,9-trioxaundecanedioic acid, and the diacid known as polyglycol diacid, which can be made from the oxidation of low molecular weight polyethylene glycol. Currently, aliphatic polyesters are among the preferred absorbable polymers for use in making the implants according to the present invention. Aliphatic polyesters can be homopolymers, copolymers (random, block, segmented, tapered blocks, graft, triblock, etc.) having a linear, branched or star structure. Suitable monomers for making aliphatic homopolymers and copolymers may be selected from the group consisting of, but are hot limited, to lactic acid (both L- and D-isomers), lactide (including L-, D-, and meso-lactide), glycolic acid, glycolide, ε-caprolactone, p-dioxanone (1,4-dioxan-2-one), trimethylene carbonate (1,3-dioxan-2-one), and combinations thereof. Aliphatic polyesters can be homopolymers, copolymers (random, block, segmented, tapered blocks, graft, triblock, etc.) having a linear, branched or star structure; alternately they can be a component of a cross-linked network. It is to be understood that exemplary bioabsorbable, biocompatible polymers may be generally synthesized by a ring-opening polymerization of the corresponding lactone monomers or by polycondensation of the corresponding hydroxy-acids, or by combinations of these two polymerization methodologies. Thus as used herein, the term “polyglycolide” is understood to include polyglycolic acid. Further, the term “polylactide” is understood to include polymers of L-lactide, D-lactide, meso-lactide, blends thereof, and lactic acid polymers and copolymers in which other moieties are present in amounts leas than 50 mole percent. Other aliphatic polyesters that may provide utility include the hydroxybutyrates and polyhydroxyvalerates.

The second part is a long pincher member 64 having a tip 66 at one end, a shaft 68, a handle 70 at the other end of the shaft 68, and a slot 72 in the body of the long pincher member 64 that is positioned between the tip 66 and the handle 70. The handle 70 of the long pincher member 64 is bulky, such that it can be gripped securely by tweezers-, forceps or other gripping devices. The long pincher member 64 is sufficiently long (i.e., roughly 5-6 cm) so that the end of the shaft 68 with the handle 70 projects into the vaginal vault 48 (not shown) when the short part 58 of the arterial clip 56 is positioned at the uterine artery 52 (not shown). The tip 66 of the long pincher member 64 and the hook 60 of the short pincher member 58 are shaped such that the hook 60 can securely latch over the tip 66. Further, the slot 72 of the long pincher member 64 and the lip 62 of the short pincher member 58 are shaped such that the lip 62 may be inserted into the slot 72. Overall, the surfaces of the long pincher member 64 are smooth and shaped to be easily withdrawn through a small opening in the vaginal wall 50 (not shown).

Both the short-pincher member 58 and the long pincher member 64 may be provided with pins 74, 76, respectively, to receive pressure from a forceps, while allowing the arterial clip 56 to rotate about the axis formed by the pins 74, 76 without moving the short pincher member 58 or long pincher member 64 relative to each other. This arrangement is explained more fully in relation to FIG. 10.

The third piece is a wire 78 that is long enough to extend through most of the length of the long pincher member 64 and has, a hooked end 80 with a tip 82. The long pincher member 64 has an interior bore (not shown) to receive the wire 78, one end of the bore being within the slot 72. The short pincher member 58 also has an interior bore (hot shown) to receive the wire 78 within an opening (not shown) at the end of the lip 62. Turning again to the long pincher member 64, the handle 70 of the long pincher member 64 may have a second bore (not shown) positioned to receive the tip 82 of the hook 80 that is turned forward along the wire 78.

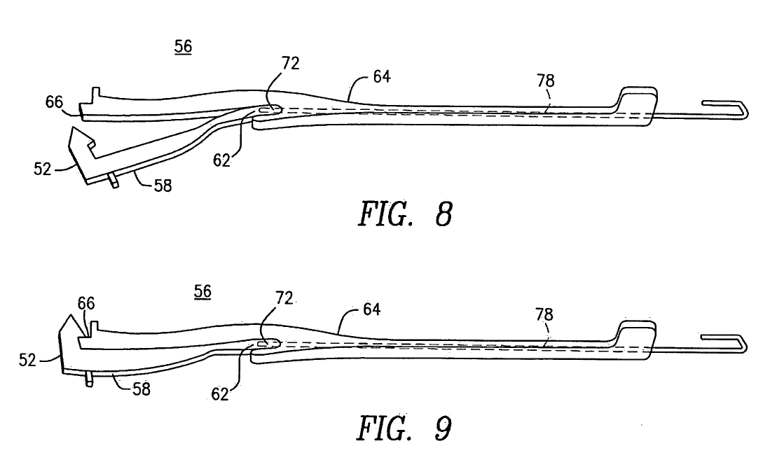

FIG. 8 shows the arterial clip of FIG. 7, assembled and in its open position. As can be seen, the lip 62 of the short pincher member 58 fits into the slot 72 of the long pincher member 64. The wire 78 extends through the body of the long pincher member 64 and into the lip 62 of the short pincher member 58. Depending on the shapes of the lip 62 and slot 72, the wire 78 could extend through the lip 62 and forward [back] into the body of the long pincher member 64.

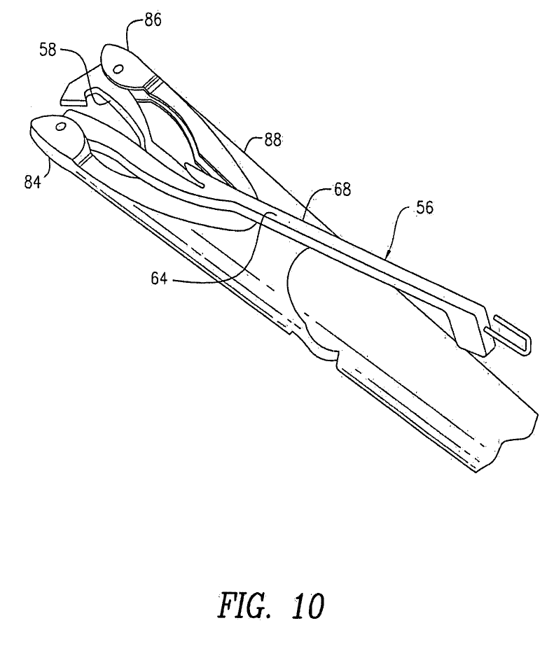

FIG. 9 shows the arterial clip 56 of FIG. 7, assembled and in its closed position. The hook 52 of the short pincher member 58 is securely latched over the tip 66 of the long pincher member 64, with the lip 62 of the short pincher member 58 and the wire 78 remaining in the positions shown in FIG. 8.

FIG. 10 shows the arterial clip 56 being gripped between opposed tongs 84, 86 of a forceps 88. In such an arrangement, it can be seen that the shaft 68 of the long pincher member 64 must lie outside of the body of the forceps 88 while the short pincher member 58 and the long pincher member 64 are held by the tongs 84, 86 of the forceps 88. Such a position is allowed by rotation of the arterial clip 56 about the axis formed by the aforementioned pins 74, 76 (see, e.g., FIG. 7).

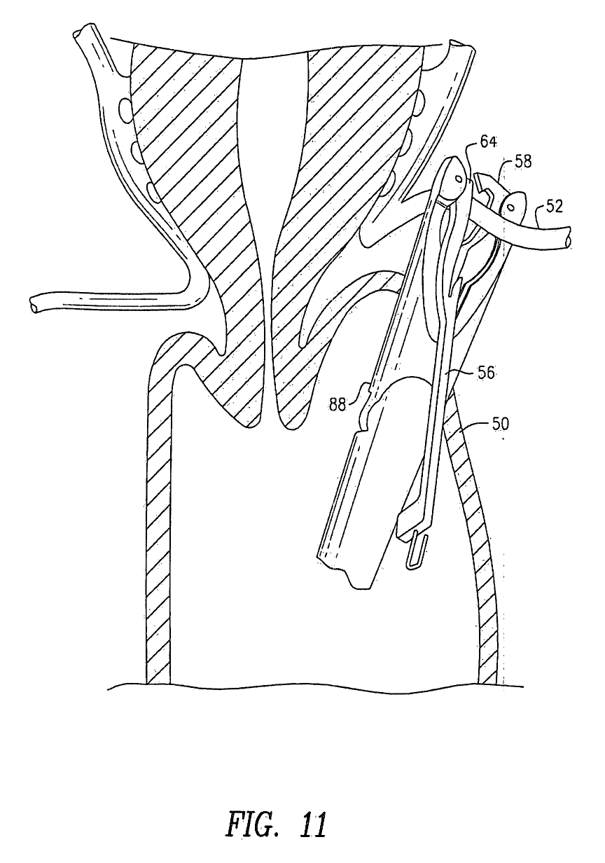

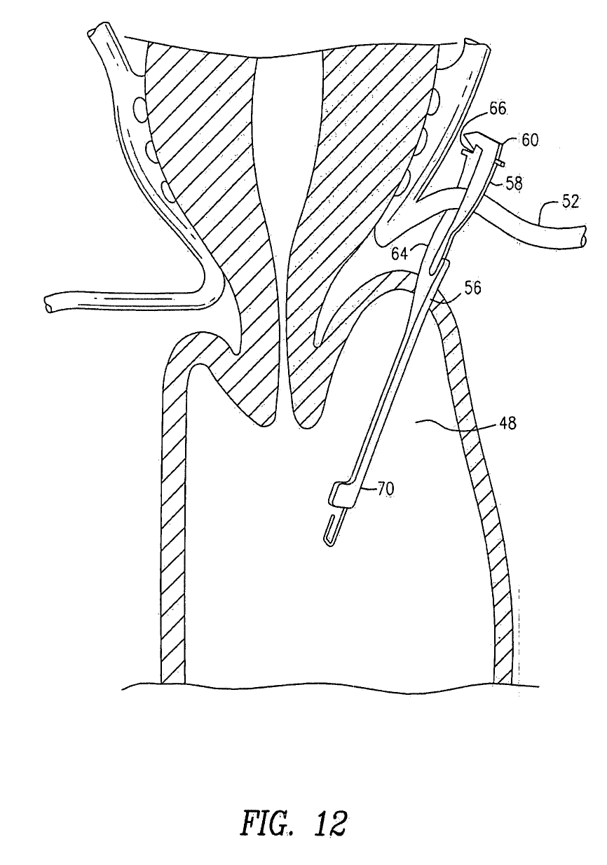

FIGS. 11 through 13 show steps in the operation of the arterial clip 56. First, an incision (not shown) is made in the vaginal wall 50 and the uterine artery 52 is dissected. The opened arterial clip 56 is manipulated, using the forceps 88, until the short pincher member 58 is on one side of the uterine artery 52 and the long pincher member 64 is on the other side of the uterine artery 52, as shown in FIG. 11.

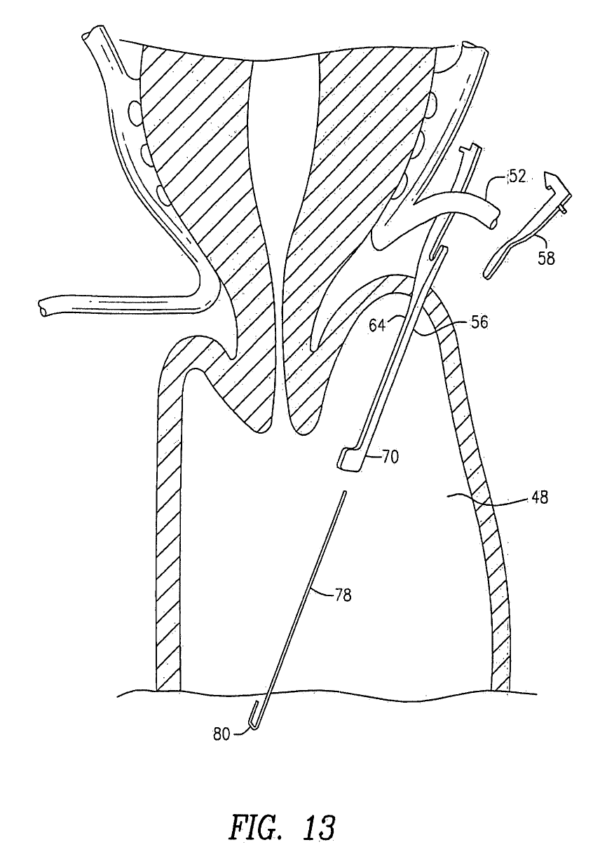

Referring to FIG. 12, the short pincher member 58 and the long pincher member 64 are pressed together, causing the hook 60 of the short pincher member 58 to catch the tip 66 of the long pincher member 64, thus locking the short pincher member 58 and the long pincher member 64 together in the closed position. When the arterial clip 56 is in this closed position, blood flow through the uterine artery 52 is blocked. The clip 56 is, then released and forceps 88 removed, making sure that the handle 70 of the arterial clip 56 remains within the vaginal vault 48.

The arterial clip 56 remains in the closed position for a period of time needed to degenerate or kill the fibroid without killing the adjacent tissue. The arterial clip 56 is then opened by gripping the handle 70 with tweezers or a similar gripping implement, gripping the hooked end 80 of the wire 78 with another implement, and withdrawing the wire, 78 from the arterial clip 56 so that the short pincher member 58 and long pincher member 64 are no longer-secured, to each other. The wire 78 is removed from the vaginal vault 48, and the short pincher member 58 of the arterial clip 56 is separated from the long pincher member 64 by gently shaking the long pincher member 64. Separating the short pincher member 58 and the long pincher member 64 relieves pressure on the uterine artery 52 and allows normal blood flow to resume. The long pincher member 64 is then pulled back through the incision and removed from the vaginal vault 48. The incision is then closed. The short pincher member 58 remains in the body, where it degrades and is absorbed.

During the procedure, the position of the arterial clip 56 relative to the uterine artery 52 may be determined by the same techniques that were identified above with respect to the arterial clip 10. Adaptations of suitable techniques for use with the arterial clip 56, will be apparent to a person skilled in the application of such techniques to surgical procedures.

It should be understood that the embodiments described herein are merely exemplary and that a person skilled in the art may make many variations and modifications thereto without departing from the spirit and scope of the present invention. For example, in the first arterial clip 10, the branches 14, 16 could be biased apart from each other and, instead of the expander 22, a collar (not shown) could be placed around the outside of the clip. The arterial clip 10 could be re-dimensioned such that the collar would cause then clip to close when it was passed over the bent sections 32, 34 or 40, 42 and to open when it passed over straight sections 28, 30 or 36, 38. With respect to the second arterial clip 56, the hook-and-tip mechanism discussed can be replaced by a ratcheting lock such as those used in some cable ties or by any other mechanism that will hold, the ends of the short pincher member and the long pincher member together while allowing the two pincher members to separate when the lip 62 is separated from the slot 72. Instead of using a short pincher member 58, a longer pincher member could be used, such that the ends of both pincher members of the device remain within the vaginal vault. In such an arrangement, restraining devices other than the wire 78 could be used to restrain the lip 62 within the slot 72. All such variations and modifications, including those discussed above, are intended to be included within the scope of the invention, which is described, in part, in the claims presented below.

We claim:

1. A device for degenerating a fibroid, comprising a pincher mechanism capable of pinching a uterine artery with a force sufficient to substantially obstruct blood flow therethrough, said pincher mechanism being sized and shaped so as to access a uterine artery through a wall of a patient's vaginal vault.

2. The device of claim 1, wherein said device is sized and shaped so as to be insertable through an incision in a vaginal wall of a female patient and maneuverable into close proximity with a uterine artery of the female, patient.

3. The device of claim 2, wherein said pincher mechanism includes a first pincher member and a second pincher member, said pincher members being movable toward each other to a closed position and away from each other to an open position.

4. The device of claim 3, wherein said first pincher member and said second pincher member each have a respective, free end and a respective end opposite said respective free end, said respective free ends being biased such that they press against each other in said closed position.

5. The device of claim 4, further comprising a solid body pressed between said first, and second pincher members and slidable relative thereto between a first position, in which said solid body allows said respective free ends of said first and second pincher members to assume said closed position, and a second position, in which said solid body urges said respective free ends of said first and second pincher members into said open position.

6. The device of claim 5, wherein said first pincher member has a bent section that substantially conforms to the shape of at least a portion of said solid body, said bent section defining said first position.

7. The device of claim 6, wherein said first pincher member has a straight section adjacent said bent section, said straight section defining said second position.

8. The device of claim 7i wherein said solid body is slidable relative to said first and second pincher members between said second position and a third position, in which, said solid body allows said respective free ends of said first and second pincher members to assume said closed position.

9. The device of claim 8, wherein said solid body is slidable relative to, said first and second pincher members between said third position and a fourth position, in which said solid body urges said respective free ends of said first and second pincher members into said open position.

10. The device of claim 3, wherein said first pincher member has a proximal end and a distal end, and said second pincher member has a proximal end and a distal end, said proximal end of said second pincher member being pivotally attached to said first pincher member intermediate said proximal and distal ends thereof, whereby said distal end of said second pincher member is pivotable toward and away from said distal end of said first pincher member.

11. The device of claim 10, wherein said distal end of said second pincher member is pivotable between an open position, in which said distal end of said second pincher member is spaced from said distal end of said first pincher member, and a closed position, in which said distal end of said second pincher member is in releasable engagement with said distal end of said first pincher member.

12. The device of claim 11, wherein said proximal end of said second pincher member is removably received in a slot formed in said first pincher member between said proximal and distal ends thereof.

13. The device of claim 12, wherein said second pincher member is releasably attached to said first pincher member by a wire extending through said first pincher member and into said second pincher member.

14. The device of claim 13, wherein said distal end of said second pincher member includes a hook which is sized and shaped so as to releasably engage said distal end of said first pincher member.

15. The device of claim 14, further comprising a first pin on said distal end of said first pincher member and a second pin on said distal end of said second pincher member, said pins being located such that said hook is moved into engagement with said distal end of said first, pincher member when pressure is applied to both of said pins.

16. The device of claim 10, wherein said second pincher member is made of a biodegradable material.

17. A method for degenerating a fibroid, comprising the steps of:
positioning a first pincher member on one side of a uterine artery and a second pincher member on an opposite side of the uterine artery; and
moving the first and second pincher members toward each other so as to pinch on the uterine artery, thereby substantially obstructing the flow of blood therethrough.

18. The method of claim 17, further including the step of pinching the uterine artery for a time that is sufficient to degrade the fibroid.
